(19) United States
(12) Patent Application Publication
(10) Pub. No.: US 2007/0129568 A1
(43) Pub. Date: Jun. 7, 2007

(54) Ionic liquids

(75) Inventors: Scott Flanagan, Atlanta, GA (US); Andrew T. Hunt, Atlanta, GA (US)

Correspondence Address: nGimatCo.;MICROCOATING TECHNOLOGIES, INC., 5315 PEACHTREE INDUSTRIAL BLVD, ATLANTA, GA, 30341-2107, US

(73) Assignee: nGimat, Co.

(21) Appl. No.: 11/607,138

(22) Filed: Dec. 1, 2006

Related U.S. Application Data
(60) Provisional application No. 60/748,002, filed on Dec. 6, 2005.

(51) Int. Cl. C07C 51/41; C07C 51/16; C07F 5/00; C07F 9/22; C07F 7/00; C07F 15/00

(52) U.S. Cl. 562/410; 534/11; 562/8; 562/606; 534/16; 556/64; 556/6; 556/81; 556/147; 548/335.5; 546/347

(57) ABSTRACT

Low melting point organic liquid compounds with high boiling points are prepared by a preferred process having the J^{+x}Q_{y}(R—COO⁻)_{x-y} where x is 1 to 8, preferably 1-3, y is 0 to x−1, where R—COO⁻ is an anion selected from the group consisting of 2-ethyl hexanoate, pivalate, neodecanoate, and mixtures thereof, Q is another anion or mixture of other anions, and J^{+x} is a cation selected from cations of Groups IA, IIA, IB, IIB, IIIB, IVB, VB, VIB, VIIB, VIII and lanthanide metals, cations selected from cations of B, Si, Ge, As, Sb, Te and Po metalloids, an ammonium cation derived from ammonia or an organic amine, an organic phosphonium cation, and mixtures thereof the organic liquid compounds being substantially free of volatile organic compounds. These compounds, as liquids, are useful as low volatile organic solvents, e.g., solvents in which a variety of chemical reactions may be carried out.

The present invention is directed to ionic compounds that are liquid at temperatures suitable for chemical processing and the uses of such ionic compounds.

### BACKGROUND OF THE INVENTION

Formulation chemists face an increasing number of restrictions on which chemicals they may select to achieve an objective. The world community now recognizes that many chemicals used by the previous generation of chemists may harm the environment and human health. The Montreal Protocol, the US Clean Air Act and the Pollution Prevention Act of 1990 strongly affect formulation practices. The US Clean Air Act includes a list of 189 Hazardous Air Pollutants (HAP) selected by Congress as possible health and environmental hazards. The paint and resin coating industry extensively uses many solvents listed as HAPS. Additional constraints on the selection of solvents include cost, performance and chemical compatibility. Unfortunately, many of the organic solvents becoming excluded from use have properties that are prized for organic synthesis, and the formulation of coatings, cleaners and a wide range of other industrial and household products. In recent years, a new class of solvents known as ionic liquids (IL's) has received increasing attention as possible replacements for volatile organic solvents, due to their low vapor pressures and wide range of properties. M. J. Earle, K. R. Seddon, Pure Appl. Chem., 72 (7), 1391-1398 (2000); R. A. Sheldon, “Green Solvents for Synthesis: State of the Art,” Green Solvents for Synthesis Conference (Dechema), Bruchsal, Germany, October 3-6; L. Crowhurst, P. R. Mawdsley, J. M. Perez-Arlandis, P. A. Salter, T. Welton, Phys. Chem. Chem. Phys., 5, 2790-2794 (2003); P. G. Jessop, R. R. Stanley, R. A. Brown, C. A. Eckert, C. L. Liotta, T. T. Ngo, P. Pollet, Green Chem., 2003, 5, 123-128; J. D. Holbrey, W. M. Reichert and R. D. Rogers, Dalton Trans., 15, 2267-2271 (2004).

The generally accepted definition of an ionic liquid is a salt having a melting point or existing as a liquid below 100° C., and a room-temperature ionic liquid (RTIL) is defined as a salt having a melting point or existing as a liquid below 25° C. The low melting point of IL's allows for their use as solvents. Combined with the fact that most IL's exhibit nominal volatility and thus nominal VOC (volatile organic compound) emissions, IL's have received intense interest in the past 20-30 years as green solvents.

Many early ionic liquids were salts of the water sensitive AlCl₄⁻ anion, limiting their practical use. Examples of conventional, water stable ionic liquids include the halide, BF₄⁻ and PF₆⁻ salts of the 1-ethyl-3-methylimidazolium (emim) and 1-butyl-3-methylpyridinium (bmpi) cations.

### SUMMARY OF THE INVENTION

The present invention is directed to ionic organic compounds that are liquid at temperatures suitable for their intended use. In some cases, it is sufficient that the ionic compounds be liquid at 200° C. or below. Preferably, however, the ionic compounds are liquid at 100° C. or below, i.e. meet the generally acceptable criteria of ionic liquids. Most preferably, the ionic compounds are liquid at 25° C. or below, i.e., meet the generally accepted criteria for room-temperature ionic liquids. As ionic compounds, the compounds have a cation or cations, and an anion or anions according to the respective ionization states. The anion(s) of the compounds of the present invention have the formula: R—COO⁻, where R is a C5-C20 saturated or unsaturated carbon chain or mixture of such carbon chains. The R or Rs have sufficient resistance to crystallization to promote a low or relatively low melting point of the ionic compound(s). An example of a suitable anion is 2-ethylhexanoate, (2eh⁻). Other carboxylic acids, including, but not limited to neodecanoic acid, pivalic acid, n-pentanoic acid, oleic acid or hexadecanoic acid could also be used as the anion source.

In one embodiment of the invention, the cation(s) is a metal or metalloid ion of +1 to +8, preferably +1 to +3, valance or a mixture of such metal or metalloid ions. When associated with appropriate anions of the above formula, many such ionic compounds have relatively low melting points. Of particular interest as metal cations are Zn⁺² and Ni⁺². Both zinc(II) 2-ethylhexanoate and nickel (II) 2-ethyl hexanoate exist as liquids below 25° C. Also of interest is cobalt (II) 2-ethylhexanoate which melts below 100° C. 2-ethylhexanoate has an asymmetric or chiral carbon atom. It is believed that in compounds where a racemic mixture of 2eh-anions are present, the presence of opposite enantiomers inhibits close packing and crystallization of the molecules, thus contributing to their low melting point. Generally, the presence of highly branched and/or chiral hydrocarbon (R) chains in the anion promote low melting points in the ionic organic compounds.

Compounds in accordance with the present invention of the formula (I) J^{+x}(R—COO⁻)ₓ, where R is a C5-C20 saturated or unsaturated carbon chain or mixture of such carbon chains where x is 1-8, preferably 1-3, and which melt at or below a desired temperature for the intended purpose tend to be water insoluble and soluble with more polar organic compounds. This makes these compounds particularly valuable as solvents for chemical reactions in which the reactants have sufficient solubility in the ionic compounds and produce a desired reaction product with water solubility. In such case, the reaction product may be extracted in an aqueous phase from the ionic compound. Alternatively, as is the case for the oxidation of p-xylene to terephthalic acid, the reactant has sufficient solubility in the ionic compounds, and the product is insoluble in the ionic compound, allowing for isolation by filtration, centrifugation or gravity settling. Other forms of separation could be yielding immiscible liquid products or fractionation due to products being higher volatility.

Another advantage of the compounds of the present invention in which a metal ion or mixture of metal ions serve as the cation(s) J, is that the metal ion(s) may serve as catalysts in the reaction mixture. Metal 2-ethylhexanoate-based ILs also have the potential to simultaneously serve as both solvent and reagent. For example, using zinc 2-ethylhexanoate as a solvent for the synthesis of a volatile zinc compound such as bis(2,2,6,6-tetramethyl-3,5-heptanedionato)zinc (Zn(tmhd)₂) would allow the zinc-containing product to be removed under vacuum as a vapor, leaving the non-volatile Zn(2eh)₂ solvent behind.

As noted, an advantage of ionic compounds in liquid form as solvents is the very low volatility of such compounds. Thus, it is desired that the ionic compounds be substantially free of volatile organic solvents. For purposes of this invention, it is preferred that volatile organic solvents are present at no more than 25 wt % relative to the ionic compounds (at 100%), preferably no more than 5 wt %, more preferably no more than 1 wt %, and even more preferably no more than 0.1 wt %. While ionic compounds of formula (I) are well known, e.g., Zn and Ni 2-ethylhexanoate, these are commonly provided in solutions of organic solvents. It is difficult to produce or isolate many useful compounds of formula (I) in substantially pure, solvent-free form.

In accordance with one aspect of the present invention, ionic compounds of formula (I) are formed by reacting acids of formula R—COOH, where R is as defined above, with a support resin (SR) having multiple (n) cationic sites to provide an anion-supported intermediate (R—COO⁻)ₙ SR⁺ⁿ, and then stripping the anions from the support with an ionic compound that provides the cation(s) J⁺.

Nickel 2-ethylhexanoate, in substantially pure form, i.e., less than 25 wt % organic solvent relative to 100% nickel 2-ethylhexanoate, preferably less than 5 wt %, more preferably less than 1 wt % organic solvent, and even more preferably less than 0.1 wt % organic solvent, has not been described or characterized previously. Substantially solvent-free nickel 2-ethylhexanoate is considered an aspect of the present invention.

Use of these compounds as solvents, e.g., as solvents for organic reactions, is considered another aspect of the present invention. Novel compounds useful as ionic solvents with low VOCs are produced from metal ions of Groups IA, IIA, IB, IIIB, IVB, VB, VIB, VIIB, and VIII. In this regard, Mn and Fe cations are of particular interest. Also of interest as cation sources J are metalloids, including B, Si, Ge, As, Sb, Te and Po. Lanthanides may also provide the cations. Preferably the oxidation state of the metal or metalloid is +1 to +3; however, oxidation states up to +8 may be useful. Generally, for use in accordance with the invention, from oxidation states +4 to +8, the higher the oxidation state, the less desirable the cation.

It is necessary for purpose of this invention that at least one anion be R—COO⁻ as defined above, and it is preferred that all of the anion be R—COO⁻. However, it is contemplated that the ionic liquid may include other anions “Q” along with the R—COO⁻. Thus, a more general formula for ionic liquids in accordance with the invention is (Ia): J^{+x}Q_{y}(R—COO⁻)_{x-y} where R is a C5-C20 saturated or unsaturated carbon chain or mixture of such carbon chains, Q is another anion or mixture of other anions, x is 1-8, preferably 1-3, and y is 0 to x−1, preferably 0.

Zinc compounds are also of interest to the present invention for use as ionic solvents and aiding in chemical reactions.

Ammonium 2-ethylhexanoate, in substantially pure form, i.e., less than 25 wt % organic solvent relative to 100% ammonium 2-ethylhexanoate, preferably less than 5 wt %, more preferably less than 1 wt % organic solvent, and even more preferably less than 0.1 wt % organic solvent, has not been described or characterized previously.

Use of these compounds as solvents, e.g., as solvents for organic reactions, is considered another aspect of the present invention.

In accordance with the present invention, additional ionic compounds are formulated that may serve as solvents. Using anions, as described above, these compounds are formulated with organic ammonium cations. Examples of such ammonium cations are those derived from an organic amine selected from the group consisting of choline, ethylenediamine, caffeine, imidzole, and pyridine or quaternany ammonium ions derived from alkylation of purines such as caffeine, hypoxanthine, theobromine, purine, adenine, guanine, xanthine or uric acid. However, this list is by no means exhaustive of ammonium ions suitable as cations for the ionic liquids of the present invention. These compounds can be prepared by the ion exchange method described above, using an ammonium salt of the parent amine.

Additional compounds are formulated with organic phosphonium cations, [R₄P]⁺, such as tetrakis(hydroxymethyl)phosphonium, tetraphenylphosphonium or trihexyl(tetradecyl)phosphonium. Ammonium and phosphonium cations are desirable for applications where a metal cation might add toxicity or an unacceptable contaminant, e.g., in semiconductor, biological, or other high purity material applications.

Ethylenediamine as the source of the ammonium ion is of particular interest in that it chelates metal ions, promoting solubility of metal ion-containing compounds and, perhaps, facilitating reactions of metal ion-containing compounds.

### DETAILED DESCRIPTION OF CERTAIN PREFERRED EMBODIMENTS

Most compounds released by manufacturing processes affect the environment in some manner. It is desired to significantly reduce the amount of man-made compounds released. Using the low volatility IL's of the present invention should help minimize environmental impact and be safer due to low vapor concentrations being non-flammable. The room temperature evaporation rates of some of the ILs described in the present invention were compared to several well known compounds by measuring the rate of weight loss in a stream of 25° C. nitrogen relative to the rate of weight loss of n-butyl acetate under the same conditions. The results are reported in the table below.

| Solvent | Evaporation Rate (n-Butyl Acetate = 1) |
|---|---|
| acetone | 14.4 |
| methyl ethyl ketone | 5.7 |
| xylene | 0.8 |
| toluene | 1.9 |
| tetraethylene glycol | <0.01 |
| ethylene glycol | <0.01 |
| hexane | 8.9 |
| IPA | 2.5 |
| n-Butanol | 0.43 |
| H2eh | 0.003 |
| Ni(2eh)₂ | <0.001 |
| Zn(2eh)₂ | <0.001 |

The new IL's are based on a significantly different chemistry than the archetypical imidazolinium and pyridinium halides that remain liquid thanks in part to the presence of an organic cation that tends not to pack for reasons such as an asymmetric structure. The new series of IL's proposed here rely instead on the use of a packing-resistant organic anion coupled with a metal cation, ammonium cation or phosphonium cation, and unlike conventional IL's, are halogen free. As metal compounds, these new IL's have the potential to fill a dual role, both as solvents and catalysts. The compounds are typically immiscible with water, suggesting that many may share the same ease of workup associated with solvents such as [bmim][PF₆⁻], but in contrast, metal 2-ethylhexanoates are generally miscible with non-polar hydrocarbons, potentially enabling their use as solvents for performing organic transformations on both polar and non-polar organic species.

The use of asymmetric organic cations to generate RTIL's has received the majority of attention in the literature to date. The organic cations commonly reported in the literature of IL's are most typically ammonium salts, frequently of aromatic amines. Described herein are anionic alternatives based on the chemistry of long chain carboxylate salts, most particularly the salts of 2-ethylhexanoic acid (H2eh). H2eh is a chiral molecule, and the asymmetric carbon atom is marked with an asterisk in the figure below. The 2eh⁻ anion has a greater degree of asymmetry than emim (emim is achiral due to a plane of symmetry), and like bmpi, has a long alkyl chain and charged head group. Certain metal and non-metal salts of a racemic mixture of H2eh function as IL's. In fact, the presence of opposite enantiomers of the anion together in the same salt is likely to be an additional factor interfering with the packing of ions to form a crystal or solidify.

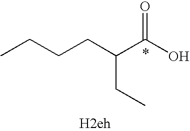

The parent acid, 2-ethylhexanoic acid, is an inexpensive commodity chemical. The primary applications for H2eh include its use as an intermediate in the production of paint and varnish driers, its conversion to esters for use as plasticizers, its use as an intermediate in pharmaceutical manufacture, and its use as a fuel and lubricant additive.

Various metal 2-ethylhexanoates (M−2eh) are commercially available, but the majority of them are not sold in analytically pure form, but rather they are sold in solution, typically in a high-boiling hydrocarbon solvent. Complete removal of the high-boiling solvent typically results in thermal degradation of the material, with significant loss of solubility. Other conventional means of separation prove ineffective; crystallization generally fails (no doubt in part due to the fact that many metal 2-ethylhexanoates are liquids at room temperature or even at reduced temperature), while chromatography on standard stationary phases usually results in adsorption of the metal ion, with concomitant exchange for surface protons. One common means of synthesizing metal 2-ethylhexanoates involves the reaction of the pure metal or an inorganic metal source such as the oxide, hydroxide, carbonate, bicarbonate or similar material with 2-ethylhexanoic acid. However, this route presents difficulties in obtaining the pure metal 2-ethylhexanoates; if an excess of 2-ethylhexanoic acid is used, it is difficult to remove from the final product for the reasons stated above. On the other hand, if an excess of metal source is used, so called overbased materials can be obtained, which are believed to involve the formation of nanoparticles of metal oxides, hydroxides, etc., having surface layers of 2-ethylhexanoate anion, rendering them difficult to remove as contaminants because they are soluble in organic solvents. Finally, the temperatures required to drive such reactions are often sufficient to cause degradation of the organic components, resulting in high-boiling organic impurities that can't be removed. The formation of such impurities is no doubt enhanced by the presence of large amounts of metal in various forms, which can serve to catalyze undesired side reactions. The few analytically pure metal 2-ethylhexanoates that are commercially available are solids at room temperature.

Salts of pivalic acide and neodecanoic acid are other organic compounds that form metal, metalloid, ammonium, and phosphonium salts useful as ionic liquids. Though these do not have a chiral carbon atom, their structure prevents close packing or inhibits strong intermolecular interactions, thereby promoting their salts being liquid at relatively low temperatures.

Certain of the organic liquids in accordance with the invention have low-toxicity relative to conventional organic solvents, such as toluene. The anions of the present invention, such as 2-eh, are demonstrably less toxic than toluene. Metal cations, such as Zn⁺² may introduce some level of toxicity. Certain non-metallic cations, such as ammonium or quaternary ammonium ions derived from the purine family, may provide ionic liquids useful as solvents with substantially no toxicity. These may be particularly important in processing materials for very high purity requirements such as use as pharmaceuticals or other products with direct human contact. Alternatively IL complexed metals that are benign or required by the body can be used such as Zn, Ca, Mg, Na, Ba, Bi and Fe.

Certain solutes are fully miscible with ionic liquids in accordance with the invention and these may be dissolved in the ionic liquid to any extent determined desirable for the application of the solution. An ionic liquid may be used as solvent even for solutes that have limited solubility in the ionic liquid, as the solute and/or product of reaction of the solute may be easily separated from the ionic liquid, making it possible to reuse the organic liquid. For practicality, however, it is preferred that a primary solute be present in solution within the organic liquid at least 1 wt % relative to the organic liquid, preferably at least 5 wt % and in some cases at least 10 wt %.

### EXAMPLE 1

In order to circumvent the problems associated with preparing metal 2-ethylhexanoates by conventional routes, samples were prepared using ion-exchange chromatography. Using a strongly basic anion exchange resin, a series of metal 2-ethylhexanoates were prepared in analytically pure form using a strongly basic ion exchange resin (Lewatit Monoplus MP 500). The resin was received in the Cl⁻ form, and was converted to the OH⁻ form by soaking in 1M NaOH for ˜2 hours. The resin was checked for residual Cl⁻ by adding a portion of the supernatant to a silver nitrate solution, with a white precipitate indicating residual chloride. Additional soaking in NaOH was continued until no Cl⁻ was detected. The resin was then washed with water and soaked in a 1 M H2eh/MeOH solution. A Pyrex Michel-Miller column 300 mm in length and with a 15 mm ID was filled with resin that was previously soaked in a 1 M solution of 2-ethylhexanoic acid in methanol. The column was then rinsed with methanol for 15 minutes at a flow rate of 5 mL/min, acetone for 45 minutes at a flow rate of 5 mL/min and pentane for 20 minutes at 5 mL/min. The column was then opened to remove any trapped air and to add more resin to fill the void created by settling of the resin. The column was then rinsed for 10 minutes with acetone at 5 mL/min, then methanol for 40 minutes at 5 mL/min.

A 0.20 M solution of the M(NO₃)ₓ was pumped through the column at 2 mL/min. In the case of colored compounds, an extraction test was performed on the product using water and pentane, when the pentane layer was colorless after the addition of product the collection was stopped. In the case of uncolored solutions the product was collected until the addition of water no longer resulted in a precipitate.

After collection, the column was rinsed with methanol for 1 hour at 1 mL/min, a 1 M solution of NaOH for 30 minutes at 5 mL/min, water for 30 minutes at 5 mL/min, methanol for 30 minutes at 5 mL/min, then a 1M solution of 2-ethylhexanoic acid for 1 hour at 5 mL /min. The column was then allowed to sit overnight in that solution to prepare it for the next synthesis.

The collected product was added to a 500 mL separatory funnel; 50-100 mL of pentane was added, then water was added. In the case of uncolored compounds, a small amount of the aqueous /methanol layer in the separatory funnel was removed and tested with additional water to ensure that complete recovery of the product. If the sample produced a white precipitate more water was added to the funnel, it was shaken, then allowed to sit for additional time. The organic layer was then collected and the solvent removed via a rotary evaporator at room temperature for ˜1 hour and then at 100° C. for ˜2 hours.

Chromium (III) 2-ethylhexanoate was obtained as a deep purple rubbery solid, indicating the possibility of a polymeric structure. Since the elemental analysis is consistent with the formulation Cr(2eh)₃, it is unlikely that any additional ligands are involved in forming polymer linkages between metal atoms. We postulate that the 2eh⁻ anion is involved in some type of bridging coordination between adjacent metal centers, giving rise to a polymeric structure. Yttrium (III) 2-ethylhexanoate was obtained as a white waxy solid, as were gallium (III) 2-ethylhexanoate and aluminum (III) 2-ethylhexanoate.

Nickel (II) 2-ethylhexanoate and zinc (II) 2-ethylhexanoate were obtained as liquids at room temperature. Zn(2eh)₂ was analyzed for C, H and O content to check agreement with the theoretical value and rule out contamination with organic liquids (tht: 54.6 wt % C, 8.6 wt % H, 18.2 wt % O, found: 53.4 wt % C, 8.4, wt % H, 18.8 wt % O). Good agreement was obtained for the wt % C, wt % H and the wt % O. In addition, GC/MS of the 2-ethylhexanoic acid starting material gave a single peak with the expected mass spectrum of H2eh with a molecular ion peak at m/z=144. An attempt was made to determine the freezing point of these liquids using low temperature DSC, however, no definite freezing transition has been observed. The liquid thickened until it no longer flowed, but did not have a freezing exotherm.

Characteristics of Zn, Ni, and Co 2-ethylhexanoate are as follows:

Zn(2eh)₂.x H₂O Prepared by ion exchange. E.A. Found (Calc. for C₁₆H₃₀O⁴Zn.0.4H₂O); C, 53.45 (53.46), H, 8.41 (8.65). FTIR (KBr): 2960 (m), 2940 (m), 2880(m), 1630 (s), 1590 (s), 1555 (s), 1470 (s), 1430 (s), 1400 (w), 1330(w), 1130 (w), 1120 (w), 808 (w), 764 (w), 739 (w) cm⁻¹. ¹H NMR (benzene-d₆): δ=2.31 (m, 1H, CH), 1.65 (m, 2H, CH₂), 1.35 (m, 6H, CH₂), 0.93 (t, 3H, CH₃), 0.88(t, 3H, CH₃). ¹³C NMR (benzene-d₆): δ=187.53 (COO), 50.54 (CH), 32.99 (CH₂), 30.48 (CH₂), 26.67 (CH₂), 23.35 (CH₂), 14.54 (CH₃), 12.51 (CH₃).

Ni(2eh)₂.x H₂O Prepared by ion exchange. E.A. Found (Calc. for C₁₆H₃₀O₄Ni-.0.7H₂O); C, 53.64 (53.73), H, 8.88 (8.85). FTIR (KBr): 3660 (w), 3612 (w), 2962 (s), 2937 (s), 2875 (m), 2870 (m), 1687 (m), 1614 (s), 1587 (s), 1464 (m), 1417 (s), 1319 (w), 1253 (w), 1220 (w), 1120 (w), 895 (w), 812 (w), 769 (w), 748 (w), 700 (w), 673 (w), 563 (w) cm⁻¹.

Co(2eh)₂.H₂O Prepared by ion exchange. E.A. Found (Calc. for C₁₆H₃₀O₄Co.H₂O); C, 52.75 (52.89), H, 8.69 (8.88). FTIR (KBr): 3647 (w), 3614 (w), 2974 (m), 2947 (m), 2879 (m), 2871 (m), 1695 (w), 1596 (m), 1469 (m), 429 (m), 1323 (w), 1238 (w), 1219 (w), 1157 (w), 1118 (m), 954 (w), 935 (w), 900 (w), 810 (m),766 (w), 734 (w), 692 (m), 586 (m), 444 (w) cm⁻¹. DSC: M.P. 73.5° C., ΔH_{fus}=40.9 J/g.

### EXAMPLE 2

Using the ion exchange procedure described in Example 1, choline 2-ethylhexanoate (ch(2eh).xH₂O) and ethylenediamene 2-ethylhexanoate (H₂en(2eh)₂) were also prepared. Characteristics of these compounds are as follows:

ch(2eh).x H₂O Prepared by ion exchange. EA Found (Calc. for C₁₃H₂₉O₃N.0.7H₂O): C, 60.11(60.10), H, 11.89 (11.78). FTIR (KBr): 3197 (s, b), 3030 (s), 2964 (s), 2939 (s), 2875 (s), 2868 (s), 1583 (s), 1489 (s), 1467 (s), 1402 (s), 1315 (s), 1257 (w), 1211 (w), 1144 (m), 1095 (s), 1045 (m), 1011 (w), 960 (s), 924 (w), 893 (w), 870 (w), 806 (w), 775 (w), 737 (w), 677 (w), 640 (w), 561 (w) cm⁻¹. ¹H NMR (methanol-d₄): δ=4.02 (m, 2H, CH₂O), 3.52 (m, 3H, CH₂N⁺R₃), 3.35 (s, 1H, ROH), 3.24 (s, 9H, (CH₃)₃N⁺R), 2.10 (m, 1H, CH), 1.54 (m, 2H, CH₂), 1.33 (m, 6H, CH₂), 0.90 (m, 6H, CH₃). ¹³C NMR (methanol-d₄): δ=184.86 (COO), 69.17 (CH₂NR₃), 57.19 (CH₂OH), 52.47 (CH), 34.31 (CH₂), 31.74 (CH₂), 27.65 (CH₂), 24.12 (CH₂), 14.70 (CH₃), 13.11 (CH₃).

H2en(2eh)₂ A 100 mL beaker, containing 25 mL ether and 3.0 g (0.05 mol) of ethylenediamine, is placed in an ice-bath and a solution of 14.4 g (0.1 mol) 2-ethylhexanoic acid in 10 mL ether is added, with stirring at such a rate as to prevent boiling of the ether. The solution is left to crystallize for a week. The crystals were dried under vacuum at room temperature for 2 hours. Yield was 15.0 g (85%) of yellowish-white needles. M.P. 43.9° C. E.A Found (Calc. for C₁₈H₄O₄N₂): C, 62.06 (62.03), H, 11.59 (11.57), N, 8.06 (8.04). FTIR(KBr): 2972 (s), 2937 (s), 2879 (s), 2873 (s), 2632 (s), 2547 (s), 2187 (m), 1637 (s), 1533 (s), 1452 (s), 1415 (s), 1319 (s), 1257 (m), 1249 (w), 1209 (w), 1137 (w), 1114 (w), 1078 (w), 1024 (w), 917 (w), 891 (w), 867 (w), 809 (m), 787 (m), 756 (w), 731 (w), 640 (m), 542 (w), 455 (m) cm⁻¹. ¹H NMR (benzene-d₆): δ=7.38 (br s, 3H, RNH₃⁺). 2.86 (br s, 2H, RCH₂N), 2.31 (br m, 1H, CH), 1.75 (m, 2H, CH₂), 1.48 (m, 6H, CH₂), 1.03 (t, 3H, CH₃), 0.94 (t, 3H, CH₃). ¹³C NMR (benzene-d₆): δ=182.64 (COO), 50.01 (CH), 40.89 (CNH₃⁺), 33.08 (CH₂), 30.81 (CH₂), 26.62 (CH₂), 23.58 (CH₂), 14.51 (CH₃), 12.73 (CH₃).

The discovery of the new room temperature ionic liquid (RTIL) salts was unexpected. RTIL's are distinguished by their very low vapor pressure (hence nominal VOC emissions) and by their unusual solvent properties that combine high dielectric (due to charge separation) with the characteristics of non-polar organics (due to the presence of long chain organics). Archetypical ionic liquids such as 1-methyl-3-ethylimidizolium hexafluorophosphate and 1-butylpyridinium nitrate contain large, unsymmetrical organic cations paired with an inorganic anion. Our new 2-ethylhexanoate based ionic liquids have, in contrast, large unsymmetrical organic anions paired with an inorganic cation. To our knowledge, these are the first RTIL's of this type to have ever been reported. Other metal 2-ethylhexanaote based RTIL's remain to be synthesized. In addition, many of the already known M-2eh salts may have melting points below 100° C., thus still qualifying as ionic liquids (the generally accepted definition for an ionic liquid is a salt that melts below 100° C.), but need to be made in the correct manner. Gallium (III) 2-ethylhexanoate and aluminum (III) 2-ethylhexanoate have also been prepared, but were not soluble in a sufficient number of solvents to allow Hansen parameters to be reliably calculated.

Two additional room temperature ionic liquids have been prepared by pairing organic cations with the 2-ethylhexanoate anion: choline 2-ethylhexanoate and ethylenediamine di-2-ethylhexanoate. Again other pairings will also yield IL and RTIL.

The metal 2-eh compounds that have so far been prepared and also have been tested for solubility in a range of 43 organic solvents, and from this data Hansen parameters have been calculated (Table I).

TABLE I
Hansen solubility parameters for tested M-2eh compounds.

| Name | δ_{D} | δ_{P} | δ_{H} | Rₒ |
|---|---|---|---|---|
| chromium(III)2-ethylhexanoate | 16.8 | 2.2 | 1.2 | 6.0 |
| yttrium(III)2-ethylhexanoate | 16.7 | 3.7 | 0.0 | 6.8 |
| cobalt(II)2-ethylhexanoate monohydrate | 23.0 | 0.0 | 10.6 | 20.0 |
| copper(II)2-ethylhexanoate | 15.7 | 7.3 | 9.7 | 12.4 |
| nickel(II)2-ethylhexanoate | 14.4 | 0.1 | 11.5 | 15.5 |
| zinc(II)2-ethylhexanoate | 14.6 | 5.8 | 10.4 | 13.0 |

Several trends are evident from the data in Table I. The first two table entries, Cr(2eh)₃ and Y(2eh)₃ share the +3 oxidation state, and their Hansen parameters are of similar magnitude, but differ markedly from the other members of the table. Furthermore, the final three entries, Cu(2eh)₂, Ni(2eh)₂ and Zn(2eh)₂ share the +2 oxidation state, and also have similar Hansen parameters. This set differs from the +3 oxidation state set in having higher values for the hydrogen bonding parameter. This difference is likely due to the lower coordination number of the +2 complexes vs. the +3 complexes. The +3 complexes, with a probable coordination number of 6, are coordinatively saturated and cannot accept additional ligands in the form of solvent molecules. The +2 complexes, on the other hand, are possibly coordinatively unsaturated in the pure form, with a coordination number of 4 or 5, and may be able to accept up to two solvent lone pairs per metal center. Co(2eh)₂.H₂O, having one water of coordination, behaves quite differently from the other +2 metals and the +3 metals, having a high hydrogen bonding parameter and a zero polar parameter. This may indicate a highly symmetrical structure to cancel out any net dipole moments, with the water of hydration accessible to solvent for hydrogen bonding. Finally, the two group IIIA metals, Al and Ga, display very similar behavior, in that both are relatively insoluble. Al(2eh)₃ was only found to dissolve in carbon tetrachloride, and Ga(2eh)₃ was only found to be appreciably soluble in carbon tetrachloride and diethanolamine. The group IIIA elements tested behave quite differently than the IIIB (Y) and VIB (Cr) in spite of having the same oxidation state.

Ionic Liquids in accordance with the invention have a variety of utilities. Many have low toxicity or can easily be separated from reaction products generated therein. The ILs of the invention are useful in the processing of metals, oxides, phosphates, carbonates, sulfates, borates, carbides, nitrides and semiconducting materials in the form of powders or coatings. The ILs may be used in the synthesis of organic polymers, monomers, organic compounds and silanes. The ILs are functional as electrolyte/solvent compositions for batteries, fuel cells, in electroplating, and as conductive liquids. Some

Features, Advantages, and Benefits are set forth in the Table II below:

TABLE II
Features, Advantages and Benefits of Ionic Liquids

| Feature | Advantage | Benefit |
|---|---|---|
| Low vapor | Essentially zero VOC | Reduced harm to the |
| pressure | emissions | environment; reduced |
| | | occupational hazards and |
| | | deaths |
| Low vapor | Very low flammability | Reduced or eliminated risk |
| pressure | | to life and property from |
| | | fire or explosion |
| Unusual | New synthetic pathways; | Greener synthesis of |
| solvent | increased rate of many | pharmaceuticals, organic |
| properties | reactions | polymers; processing of ores, |
| | | refractories, metal oxides |
| Can form | Can be used for | Easier workup of reaction |
| biphasic | liquid—liquid extraction | products; recycling of ionic |
| systems | | liquids |
| Electrical | Can be used as | Can serve as electrolyte and |
| conductivity | conductive solvent | solvent for fuel cells, sensors, |
| | | batteries, electroplating |

Ionic liquids based on 2-ethylhexanoic acid may be used for epoxidation reactions, such as, but not limited to the synthesis of epichlorohydrin, which is a monomer used to produce epoxy resin. For example, by dissolving allyl chloride in an ionic liquid, such as, but not limited to zinc 2-ethylhexanoate, and providing an oxidant such as but not limited to pressurized air, hydrogen peroxide, or a mixture of manganese sulphate and sodium carbonate and optionally a catalyst, such as, but not limited to Jacobsen's catalyst, the allyl chloride may be epoxidized to produce epichlorohydrin. This reaction potentially occurs at room temperature, and will occur at elevated temperatures up to 250° C.

### EXAMPLE 3

Terephthalic acid is a chemical precursor for polyethyleneterephthalate (PET), a plastic commonly used, for example, in clear beverage bottles. Terephthalic acid is most commonly manufactured by oxidation of p-xylene in acetic acid, using flowing, pressurized air as the oxidizer. Typical reaction conditions are 200° C. at 15-30 atmospheres pressure of flowing air.

In this example, various conditions and reactants were run in a static air pressurized vessel. Reactants and conditions are given in the table below. Where no “P (psi)” given, air pressure is ambient.

| | | | Solvent (g) | | | Reagent (g) | | Catalyst (g) | | | |
|---|---|---|---|---|---|---|---|---|---|---|---|
| T (° C.) | P (psi) | t (m) | Zn(2eh)₂ | HOAc | H₂O | p-Xylene | H₂O₂ | NaBr | Co(2eh)₂ | Mn(2eh)₂ | % Yd |
| 200 | | 120 | 1.68 | | | 3.62 | 2.02 | 0.1097 | | | 80.6 |
| 200 | | 120 | 1.24 | | | 4.25 | 2.14 | 0.0605 | | | 20.5 |
| 200 | 100 | 60 | 12.09 | | | 4.08 | | 0.0405 | 0.0119 | 0.0463 | 4.2 |
| 200 | | 120 | 1.18 | | | 4.02 | | 0.0512 | | | 2.7 |
| 200 | 200 | 60 | 6.09 | | | 2.01 | 1.04 | 0.0226 | 0.0029 | 0.0190 | 2.5 |
| 200 | | 120 | 1.38 | | | 4.13 | | 0.0637 | 0.0324 | | 2.3 |
| 200 | 100 | 60 | 4.28 | | | 12.15 | | 0.0084 | 0.0046 | 0.0166 | 1.8 |
| 200 | 100 | 60 | 6.09 | | | 2.01 | | 0.0218 | 0.0321 | 0.0142 | 1.7 |
| 200 | 100 | 60 | 1.48 | | | 6.07 | | 0.0274 | 0.0106 | 0.0704 | 0.8 |
| 190 | 200 | 60 | 12.19 | | 1.13 | 4.13 | 2.06 | 0.0200 | 0.0085 | 0.0292 | 0.7 |
| 200 | 200 | 60 | 9.41 | | | 12.02 | | 0.0283 | 0.0060 | 0.0266 | 0.3 |
| 250 | 200 | 60 | 9.66 | | | 12.01 | | 0.0754 | 0.0069 | 0.0353 | 0.3 |
| 200 | 100 | 120 | 1.46 | | | 4.08 | | 0.0538 | 0.0596 | 0.1905 | 0.3 |
| 200 | 100 | 60 | | 6.27 | | 2.05 | | 0.0218 | 0.0029 | 0.0989 | 0.0 |
| 250 | 100 | 60 | | 6.10 | | 2.04 | 1.01 | 0.0103 | 0.0030 | 0.0098 | 0.0 |
| 150 | 200 | 60 | | 6.02 | | 2.13 | 1.00 | 0.0212 | 0.0027 | 0.0198 | 0.0 |

The low yield of product in the presence of acetic acid as a solvent is believed to be attributable to the static, rather than dynamic, nature of the reactor used. The same reactor was used for all experiments, and even in these static conditions, however, metal ethylhexanoates as the reaction medium solvents yielded product (proved superior to acetic acid). Zinc ethylhexanoate was particularly effective, particularly with the conditions listed in the top row of the table where additional savings are possible with the use of unpressurized reactors. Zinc ethylhexanoate is basically non-volatile, while acetic acid, the conventional solvent, presents the problem of evaporation and VOC release. An important aspect of the current invention is the use of metal cation complexed IL for the enhanced synthesis of compounds such as but not limited to Zn(tmhd)₂ and terephthalic

Ionic liquids have a wide range of applications in electrochemistry including electroplating and fuel cells. The chromium electroplating industry has a need for green materials, specifically with respect to the valence of the metal. Hexavalent chromium is currently used because of the limited solubility of other starting materials. If ionic liquids are employed such as imidazolium salts, trivalent chromium may be used. Additionally, chromic acids need not be used and minimal hydrogen is evolved in the working potential window helping extend the lifetime of equipment. Choline-carboxylate salts have enormous potential to overcome these cost and safety-related barriers.

Ionic liquids based on 2-ethylhexanoic acid also have the potential to be used in electroplating metal coatings on active metals, such as but not limited to titanium. Titanium is difficult to deposit well-adhered coatings onto, due to the presence of a surface oxide layer. Exposure of freshly cleaned titanium surfaces to water-based electroplating solutions can result in re-formation of an oxide or hydroxide layer due to reaction with water. By operating in an inert atmosphere and using an anhydrous ionic liquid based on 2-ethylhexanoic acid such but not limited to ethylenediamine 2-ethylhexanoate, it may be possible to electroplate on the surface of freshly cleaned titanium. By using metal-based 2-ethylhexanoates salts in pure form or mixed with other more conductive 2eh based ILs, the IL may serve as both the solvent and the electrochemical reagent, providing the source of the metal to be deposited on the reactive metal substrate.

Ionic liquids also have great potential as low-humidity polymer electrolytes in PEM (proton exchange membrane) fuel cells. Long-chain carboxylate salts with various side functionalities can act as proton conductive media at elevated (>120° C.) temperatures where perfluorinated polymers (Nafion™) requiring constant hydration fall short. Additionally, Nafion is an expensive material making PEM fuel cell technology cost prohibitive. Additionally, choline-based long chain carboxylates have strong potential to provide higher temperature/lower humidity electrolytes enabling higher temperature PEM fuel cell operation, a necessary set of conditions toward making low-cost CO tolerant fuels work.

### EXAMPLE 4

Choline 2-ethylhexanoate was used as a co-solvent in the production of SmCO₅ magnetic nanoparticles. In a three-neck round bottom flask under a hydrogen/argon atmosphere 1,2-hexadecanediol (0.122 g, 0.45 mmol), choline 2-ethylhexanoate (5 ml) and dioctyl ether (10 ml) were mixed and heated to 100° C. Samarium acetylacetonate (0.0765 g, 0.17 mmol) was dissolved in 5 ml of dioctyl ether. (Samarium acetylacetonate was previously dehydrated under vacuum at 100° C.) The solution was transferred into the 1,2 hexadecanediol solution. Oleic acid (0.06 ml, 0.2 mmol) and oleylamine (0.06 ml, 0.2 mmol) were added to the reaction mixture and the temperature was maintained at 100° C. Dicobalt carbonyl (0.172 g, 0.5 mmol) was dissolved in 10 ml of dioctyl ether, and this solution was added to the samarium solution. The mixture was heated to 270-280° C. and allowed to reflux for 30 min. The heat source was then removed, and the reaction mixture was allowed to cool to room temperature. The black product was precipitated by adding ethanol (20 ml) and separated by centrifugation. The black precipitate was dispersed in hexane (20 ml). After adding 0.02 ml of oleic acid and 0.02 ml of oleylamine, the precipitate appeared and was isolated by centrifuging. The precipitate was washed with ethanol (20 ml) and centrifuged to get the black solid product, which was washed again with ethanol (20 ml), centrifuged, and finally redispersed in hexane.

What is claimed:

1. A compound or mixture of compounds having the formula J^{+x}Q_{y}(R—COO)_{x-y} where x is 1 to 8, y is 0 to x−1, where R—COO⁻ is an anion selected from the group consisting of 2-ethyl hexanoate, pivalate, neodecanoate, and mixtures thereof, Q is another anion or mixture of other anions, and J^{+x} is a cation selected from cations of Groups IA, IIA, IB, IIIB, IVB, VB, VIB, VIIB, VIII and lanthanide metals, cations selected from cations of B, Si Ge, As, Sb, Te and Po metalloids, an ammonium cation derived from ammonia or an organic amine, an organic phosphonium cation, and mixtures thereof, said compound or mixture of compounds existing as a liquid at a temperature of 200° C. or below, said compound or mixture of compounds containing no more than about 25 wt % volatile organic solvent relative to the weight of said compound or mixture of compounds of formula J^{+x}Q_{y}(R—COO⁻)_{x-y} (at 100%).

2. The compound or mixture of compounds according to claim 1 wherein said compound or mixture of compounds exists as a liquid at a temperature of 100° C. or below.

3. The compound or mixture of compounds according to claim 1 wherein said compound or mixture of compounds exists as a liquid at a temperature of 25° C. or below.

4. The compound or mixture of compounds according to claim 1 wherein J comprises a metal selected from the group consisting of nickel (II) and cobalt (II).

5. The compound or mixture of compounds according to claim 1 wherein J^{+x} comprises an organic cation selected from the group consisting of choline, ethylenediammine, imidazolium, and pyridinium.

6. The compound or mixture of compounds according to claim 1 wherein J^{+x} comprises the ammonium cation.

7. The compound or mixture of compounds according to claim 1 containing no more than about 1 wt % volatile organic solvent relative to the weight of said compound or mixture of compounds of formula J^{+x}Q_{y}(R—COO⁻)_{x-y} (at 100%).

8. The compound or mixture of compounds according to claim 1 containing no more than about 0.1 wt % volatile organic solvent relative to the weight of said compound or mixture of compounds of formula J^{+x}Q_{y}(R—COO⁻)_{x-y} (at 100%).

9. The compound or mixture of compounds according to claim 1 wherein said compound or mixture of compound is used in the synthesis of pharmaceuticals.

10. The compound or mixture of compounds according to claim 1 wherein said compound or mixture of compounds has utility from the group consisting of processing of metals, oxides, phosphates, carbonates, sulfates, borates, carbides, nitrides or semiconductors as a powder or as a coating, the synthesis of organic polymers, monomers, organic compounds, and silanes, as electrolyte/solvent for batteries, fuel cells, or electroplating or functional as conductive liquids.

11. The compound or mixture of compounds according to claim 1 wherein x is 1-3.

12. The compound or mixture of compounds according to claim 1 wherein said compound or mixture of compounds are functional for running a chemical reaction comprising dissolving a chemical reactant or reactants in said compound or mixture of compounds to prepare a solution and reacting said reactant(s) within said solution.

13. A method of preparing a compound of the formula:
J₊ₓ(R—COO⁻)ₓ where x is 1 to 8, where R is a C5-C20 saturated or unsaturated carbon chain or mixture of such carbon chains, and J^{+x} is a cation selected from the group of a metal cation, a metalloid cation, an ammonium cation derived from ammonia or an organic amine, a phosphonium cation, and mixtures of these cations,
the method comprising
providing a support resin (SR) having multiple (n) cationic sites,
reacting said support resin with an organic acid of formula R—COOH to prepare an organic intermediate having the formula (R—COO⁻)ₙ SR⁺ⁿ, and
stripping the anions from the support with an ionic compound that provides the J⁺ cation.

14. The method according to claim 12 where J is zinc (II), nickel (II), or cobalt (II).

15. A method of preparing a solution of low volatile organic compound content comprising,
providing as an ionic solvent a compound or mixture of compounds having the formula J^{+x}Q_{y}(R—COO⁻)_{x-y} where x is 1 to 8, y is 0 to x−1, where R—COO⁻ is an anion selected from the group consisting of 2-ethyl hexanoate, pivalate, neodecanoate, and mixtures thereof, Q is another anion or mixture of other anions, and J^{+x} is a cation selected from cations of Groups IA, IIA, IB, IIB, IIIB, IVB, VB, VIB, VIIB, VIII and lanthanide metals, cations selected from cations of B, Si, Ge, As, Sb, Te and Po metalloids, an ammonium cation derived from ammonia or an organic amine, an organic phosphonium cation, and mixtures thereof, said compound or mixture of compounds existing as a liquid at temperatures of 200° C. or below, said compound or mixture of compounds containing no more than about 25 wt % volatile organic solvent relative to the weight of said compound or mixture of compounds (at 100%) and
dissolving at least one solute compound therein.

16. The method of claim 15 wherein x is 1-3.

17. The method according to claim 16 where J comprises a metal selected from the group consisting of nickel (II), zinc (II), and cobalt (II).

18. The method according to claim 16 wherein R—COO⁻ comprises an anion derived from an organic compound selected from the group consisting of pivalic acid, 2-ethylhexanoic acid and neodecanoic acid.

19. The method according to claim 16 wherein said at least one solute comprises p-xylene.

20. A method of synthesizing terephthalate comprising preparing a p-xylene solution according to claim 19 and oxidizing said p-xylene within said solution.
